(19) United States
(12) Patent Application Publication
(10) Pub. No.: US 2009/0186960 A1
(43) Pub. Date: Jul. 23, 2009

(54) DENTAL MATERIALS WITH SURFACE-FUNCTIONALIZED FILLERS

(75) Inventors: Norbert MOSZNER, Triesen (LI); Jorg ANGERMANN, Sargans (CH); Simone KLAPDOHR, Rosenheim (DE)

Correspondence Address: NIXON PEABODY LLP - PATENT GROUP, 1100 CLINTON SQUARE, ROCHESTER, NY, 14604, US

(73) Assignee: IVOCLAR VIVADENT AG, Schaan (LI)

(21) Appl. No.: 12/275,700

(22) Filed: Nov. 21, 2008

(30) Foreign Application Priority Data
Jan. 18, 2008 (EP) 08000956.6

(51) Int. Cl. A61K 6/093; C08G 77/14; C07F 5/06; C07F 7/18; C07F 5/00

(52) U.S. Cl. 523/116; 528/9; 528/33; 534/15; 556/9; 556/443

(57) ABSTRACT

The invention relates to a polymerizable composition which is characterized in that it contains at least one filler that is surface-functionalized with groups of formula (I), wherein groups of formula (I)

(A)ₐ-Z-Y—R²—SiR¹₃₋ₘ—(O—)ₘ (I),

are bonded to the filler via at least one oxygen atom that is bound to the silicon atom of the group of formula (I). The invention also relates to a process for the preparation of the composition according to the invention and its use in particular as a dental material.

This application claims the benefit of European Patent Application Serial No. 08000956.6, filed Jan. 18, 2008, which is hereby incorporated by reference in its entirety.

### FIELD OF THE INVENTION

The present invention relates to compositions, based on surface-functionalized fillers, which are particularly suitable as dental materials. The invention also relates to surface-functionalized fillers, a process for the preparation of the compositions and fillers according to the invention, and their use as dental materials for the preparation of adhesives, coatings or composites.

### BACKGROUND OF THE INVENTION

To effect the adhesion of dental materials to the tooth structure (enamel and dentine), it is known to use in the dental materials polymerizable monomers which can engage in binding interactions with hydroxylapatite or collagen, for example ethylenically unsaturated monomers which contain aldehyde, β-diketone, β-ketoester, carboxylic acid anhydride or acid groups (cf. N. Moszner, U. Salz, J. Zimmermann, Dental Materials 21 (2005) 895-910; U. Salz, S. W. Shalaby, Polymers for Dental and Orthopedic Applications, CRC Press, Boca Raton etc. 2007, 69 et seq.). Thus methacrylates which contain carboxylic acid, carboxylic acid anhydride, phosphonic acid, phosphoric acid or sulphonic acid groups, or glutaraldehyde are used as components in commercial enamel-dentine adhesives.

In addition, certain acids are also used as reaction components in dental cements, for example simple acids such as phosphoric acid as reaction partner for ZnO in phosphate cements, polyacrylic acid as reaction partner for ZnO in polycarboxylate cements, copolymers of acrylic acid and itaconic acid as reaction partner for calcium-aluminium silicate glasses in glass ionomer cements or also certain acid monomers as reaction component for calcium-aluminium silicate glasses in compomers (cf. E. C. Combe, F. J. T. Burke, W. H. Douglas, Dental Biomaterials, Kluwer Academic. Publishers, Boston etc. 1999, 211 et seq., 221 et seq., 233 et seq.; U. Salz, S. W. Shalaby, Polymers for Dental and Orthopedic Applications, CRC Press, Boca Raton etc. 2007, 49 et seq.).

Fillers, in particular silicate and non-silicate inorganic fillers, are often used to mechanically reinforce dental materials. The silicate fillers mainly used include ground glasses such as e.g. barium-silicate glasses (U.S. Pat. No. 4,220,582), strontium-silicate glasses (DE 43 23 143), lithium-aluminium silicate glasses (GB 1 488 403) and X-ray-opaque aluminium-fluorosilicate glasses, which are used primarily in methacrylate-reinforced glass ionomers (U.S. Pat. No. 5,367,002, U.S. Pat. No. 5,871,360). Pure silicon oxide fillers are likewise used in dental materials (DE 24 05 578). Also known are mixed oxides based on silicon and zirconium oxide (DE 32 47 800). In addition to mechanical reinforcement, fillers are also used to increase X-ray opacity and to set consistency and transparency. Non-silicate fillers are used in particular as X-ray contrast media, for example zirconium oxide (WO 00/69392), tantalum oxide (WO 98/13008) or yttrium oxide (DE 100 18 405). Aluminium and titanium oxide serve as opacifiers on account of their high refractive index.

By modifying their surface, various properties of fillers can be adjusted. In the case of inorganic, silicate fillers, for example, a silanization can be carried out for this. Thus, in order to set hydrophilic or hydrophobic properties, UV-absorbency and dirt-repellent properties of fillers and also to improve their suspensibility and incorporability into a plastic matrix a process is known from DE 10 2004 022 566 A1 for coating glass, glass ceramic and/or ceramic powders, in which silanes provided with specific functional groups are used as coating reagents.

In dentistry the use of fillers is known the surfaces of which are modified with polymerizable groups, with the result that the latter are bonded covalently to the polymer matrix (for example a methacrylate matrix) by copolymerization during the curing of the material. For this purpose, silicate fillers can be silanized for example with prehydrolyzed (meth)acryloxyalkyltrialkoxy silanes (cf. e.g. DE 40 29 230 for filling and fixing materials, or US 2002/0065337 for coatings). Non-silicate fillers such as e.g. zirconium oxide can be surface-modified for example by methacrylate-modified polyether carboxylic acids (U.S. Pat. No. 6,387,981) or (meth)acryloyloxyalkyl dihydrogen phosphates (U.S. Pat. No. 6,417,244).

The mechanical properties of dental materials can be improved by using such fillers. It has been found, however, that known dental materials have an adhesion to the tooth material which in many cases is not optimum.

Silicate materials surface-functionalized with aldehyde or acid groups are used in molecular biology or affinity chromatography for example to immobilize proteins and polypeptides. The preparation of SiO₂ particles or SiO₂ nanotubes functionalized with CHO groups is carried out e.g. via a reaction with aldehyde-group-containing silanes, such as e.g. trimethoxysilylbutyraldehyde or trimethoxysilylpropionaldehyde (cf M. T. Dulay et al., Analyt. Chem. 77 (2005) 4604-4610; G. MacBeath, S. L. Schreiber, Science 289 (2000) 1760-1763; W. Clarke et al., J. Chromatography A 2000 (888) 13-22; S. B. Lee et al., Science 296 (2002) 2198-2200).

### SUMMARY OF THE INVENTION

An aspect of the invention is to provide fillers which can be easily worked into various resin or polymer matrix systems and are suitable for the preparation of dental adhesives, cements, composites or coatings, have good mechanical properties and display an improved adhesion to the tooth structure.

### DETAILED DESCRIPTION OF VARIOUS EMBODIMENTS

The invention relates to a polymerizable composition which contains at least one filler that is surface-functionalized with groups of formula (I), wherein groups of formula (I)

(A)ₐ-Z-Y—R²—SiR¹₃₋ₘ—(O—)ₘ (I),

in which
R¹ represents C₁-C₁₅ alkyl, C₂-C₅ alkenyl or phenyl,
R² is missing or represents a linear or branched C₁-C₆ alkylene radical,
Y is missing or represents an ether, thioether, amide, ester or urethane group,
Z is missing or represents an at least divalent linear or branched aliphatic radical with 2 to 20 carbon atoms, which can be interrupted by one or more ether, thioether, amide or ester groups and which can contain one or more cycloaliphatic groups with at least 3 carbon atoms and/or one or more aromatic groups with at least 6 carbon atoms, an at least divalent cycloaliphatic radical with at least 3 carbon atoms or an at least divalent aromatic radical with at least 6 carbon atoms,
A represents in each case independently —COOH, —P(O)(OH)₂, —O—P(O)(OH)₂, —SO₂OH, —C(O)—O—C(O)—, —CHO, —NH—C(O)—CHO, —C(O)—CHO, —C(O)—CH₂—C(O)—CH₃, —N═C═O or —O—C(O)—CH₂—C(O)—CH₃,
a is 1 to 6 and
m is 1 to 3,
wherein R² and Z cannot both be missing,
wherein R² and Z can in each case be missing only if Y is simultaneously also missing, and
wherein a is 1 if Z is missing,
   are bonded to the filler via at least one oxygen atom that is bound to the silicon atom of the group of formula (I).

Here, the term “bonded” refers to a chemical bond, preferably a covalent chemical bond.

The detail that a radical can be interrupted by a group, such as for example an ether group, is to be understood such that the group can be inserted into the carbon chain of the radical, i.e. is bordered on both sides by carbon atoms. The number of these groups is therefore at least 1 less than the number of carbon atoms and the groups cannot be terminal. Preferred according to the invention are radicals which are not interrupted by the named groups.

If A is a divalent group, in particular —C(O)—O—C(O)—, the two terminal carbon atoms of this group are each bound to different carbon atoms of group Z. If the group of formula (I) contains more than one group A, the several groups A can each be bound to the same and/or preferably to different carbon atoms of group Z.

According to the invention, only compounds which are compatible with the chemical valence theory are contemplated.

It was surprisingly found that the compositions according to the invention, which contain a filler that is surface-functionalized with groups of formula (I), are suitable in particular as dental materials which are characterized by an improved adhesion to the tooth structure, in particular an improved adhesive shear strength to dentine and tooth enamel. Without limitation to a specific theory, it is assumed that fillers surface-functionalized with groups of formula (I) can engage in covalent bonds with hydroxylapatite and/or collagen of the tooth structure via the functional groups A. In particular, acid groups can react with hydroxylapatite and carboxylic acid anhydrides or aldehydes with collagen.

A preferably represents in each case independently —COOH, —P(O)(OH)₂, —O—P(O)(OH)₂, —SO₂OH, —CHO, —NH—C(O)—CHO, —C(O)—CHO, —C(O)—CH₂—C(O)—CH₃, or —O—C(O)—CH₂—C(O)—CH₃.

A preferred embodiment of the polymerizable composition is characterized in that
R¹ represents C₁-C₆ alkyl or phenyl,
R² represents linear or branched C₁-C₃ alkylene,
Y is missing or represents an ether, thioether, ester or urethane group,
Z is missing or represents an at least divalent linear or branched aliphatic radical with 2 to 20 carbon atoms, which can be interrupted by one or more ether, thioether, amide or ester groups and which can contain one or more cycloaliphatic groups with at least 3 carbon atoms and/or one or more aromatic groups with at least 6 carbon atoms, an at least divalent cycloaliphatic radical with at least 3 carbon atoms or an at least divalent aromatic radical with at least 6 carbon atoms,
A represents in each case independently —COOH, —P(O)(OH)₂, —O—P(O)(OH)₂, —SO₂OH, —CHO, —NH—C(O)—CHO or —O—C(O)—CH₂—C(O)—CH₃,
a is 1 to 3 and
m is 1 to 3.

R¹ particularly preferably represents C₁-C₃ alkyl, most preferably for methyl.

R² particularly preferably represents C₁-C₃ alkylene.

Y particularly preferably represents an ether or thioether group.

Z particularly preferably represents an at least divalent linear or branched aliphatic radical with 2 to 10 carbon atoms, which can be interrupted by one or more ether or ester groups and which can contain one or more cycloaliphatic groups with at least 3 carbon atoms and/or one or more aromatic groups with at least 6 carbon atoms, an at least divalent cycloaliphatic radical with at least 3 carbon atoms or an at least divalent aromatic radical with at least 6 carbon atoms.

A particularly preferably represents in each case independently —P(O)(OH)₂, —O—P(O)(OH)₂, —CHO or —NH—C(O)—CHO.

a is particularly preferably 1 or 2.

The named alkyl and alkylene radicals are preferably linear groups.

The compositions according to the invention contain at least one filler the surface of which is functionalized. Inorganic particles and fibres in particular are suitable as filler. Particulate materials with an average particle size of from 1 nm to 10 μm, preferably from 5 nm to 5 μm, are preferably used as filler. The term average particle size refers here to the average by volume.

Inorganic, preferably amorphous materials are preferred fillers. Monodisperse, nanoparticulate fillers, preferably based on SiO₂, such as pyrogenic silicic acid or precipitation silicic acid, oxides of the elements Zr, Ti, Al, Y, La, Ce and/or Yb and their mixed oxides with SiO₂ are particularly preferred. It is preferred that the filler has an average particle size of 5 to 200 nm, particularly preferably 10 to 100 nm, quite particularly preferably 10 to 50 nm.

The groups of formula (I) can generally be derived from silanes of formula (II)

(A)ₐ-Z-Y—R²—SiXₙR¹₃₋ₙ (II),

in which
X represents halogen, hydroxy, C₁-C₅ alkoxy or C₁-C₃ acyloxy,
A represents in each case independently —COOH, —P(O)(OR³)₂, —O—P(O)(OR³)₂, —SO₂OH, —C(O)—O—C(O)—, —CHO, —NH—C(O)—CHO, —C(O)—CHO, —C(O)—CH₂—C(O)—CH₃, —N═C═O or —O—C(O)—CH₂—C(O)—CH₃,
R³ represents in each case independently H or C₁-C₅ alkoxy,
n is 1 to 3
   and the remaining meanings are as defined above for formula (I).

A preferably represents in each case independently —COOH, —P(O)(OR³)₂, —O—P(O)(OR³)₂, —SO₂OH, —CHO, —NH—C(O)—CHO or —O—C(O)—CH₂—C(O)—CH₃.

X particularly preferably represents halogen or C₁-C₃ alkoxy, in particular Cl, methoxy, ethoxy or n-propoxy, most preferably methoxy.

A particularly preferably represents in each case independently —P(O)(OR³)₂, —O—P(O)(OR³)₂, —CHO or —NH—C(O)—CHO.

R³ particularly preferably represents H or C₁-C₃ alkoxy, most preferably H, methoxy or ethoxy.

Examples of silanes according to formula (II) are i.a.:

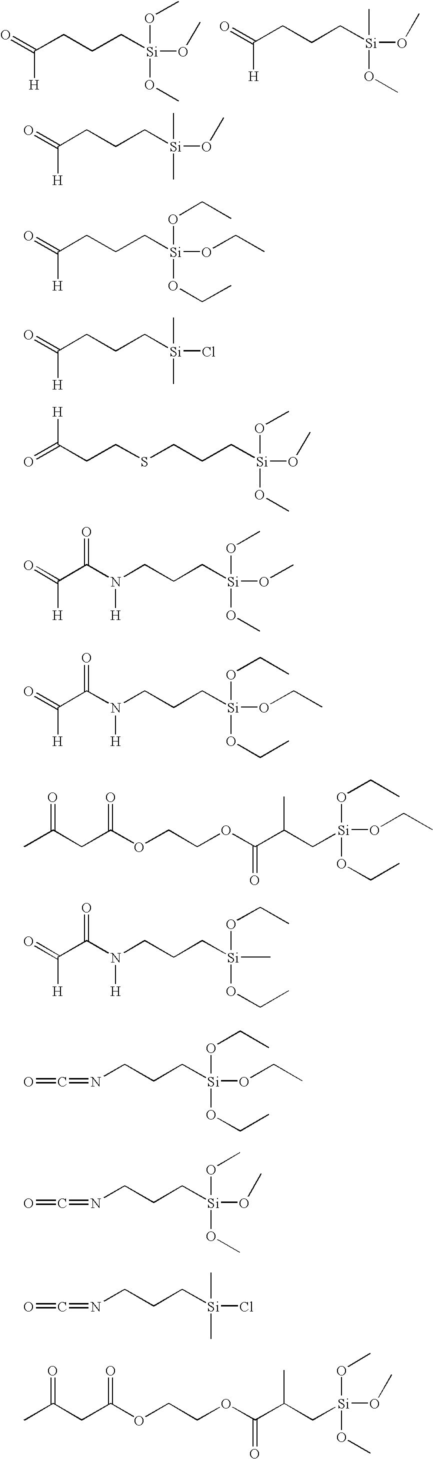

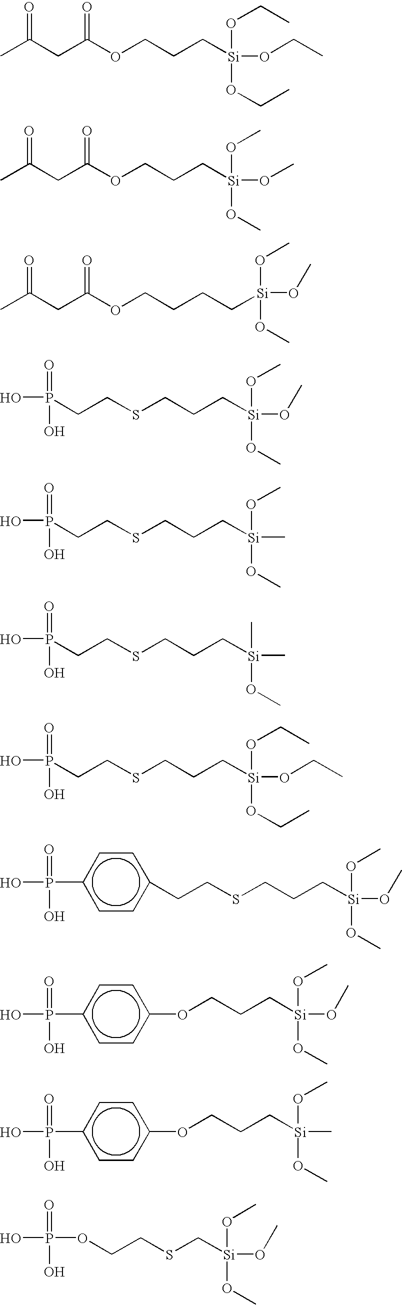

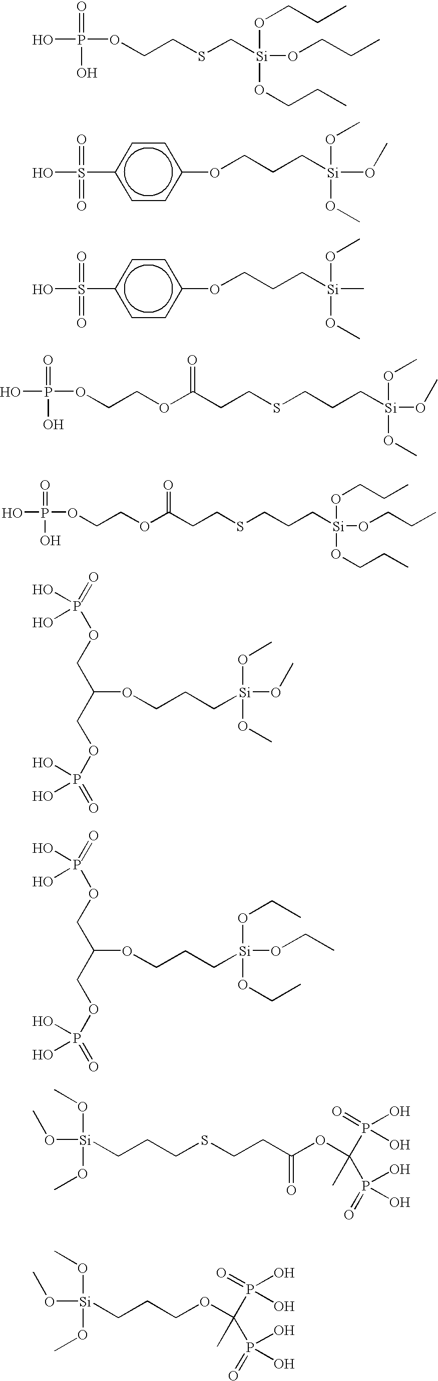

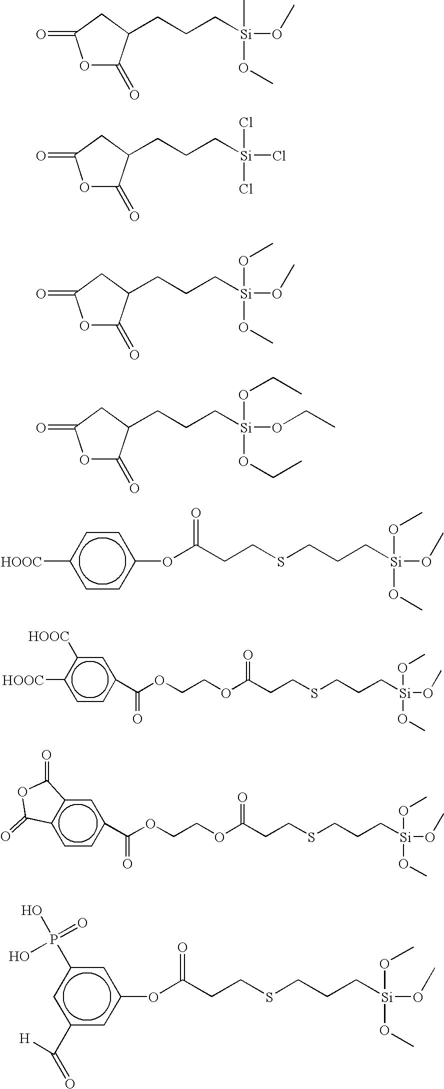

Compositions which have at least one filler that are surface-functionalized with groups of formula (I) wherein the groups of formula (I) are derived from one of the above-named silanes of formula (II) are particularly preferred according to the invention.

Some of the functionalized silanes according to formula (II) are known or commercially available. For example, the following silanes are commercially available:

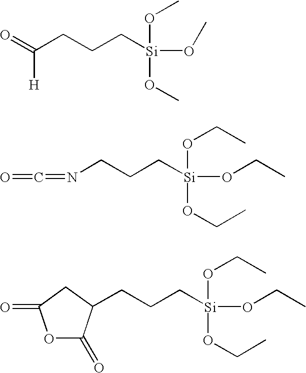

Silanes according to formula (II) can generally be prepared analogously to methods known from silicon chemistry (for example M. A. Brook, Silicon in Organic, Organometallic, and Polymer Chemistry, John Wiley & Sons Inc., New York etc. (1999), which is hereby incorporated by reference in its entirety) and organic chemistry (for example W. Walter, W. Franke, Bayer-Walter Lehrbuch der organischen Chemie, 24^{th} ed., S. Hirzel Verlag, Stuttgart and Leipzig 2004; Autorenkollektiv, Organikum, 21^{st} ed., Wiley-VCH, Weinheim etc. (2001), which is hereby incorporated by reference in its entirety).

A synthesis method is for example the bonding of Si—H— and vinyl-group-containing compounds by hydrosilylation:

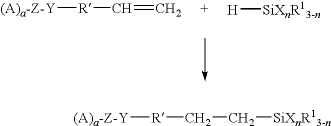

Specific example for the preparation of an aldehyde-group-containing silane: hydrosilylation of allyl alcohol with trimethoxysilane followed by oxidation of the primary OH-group to form an aldehyde group:

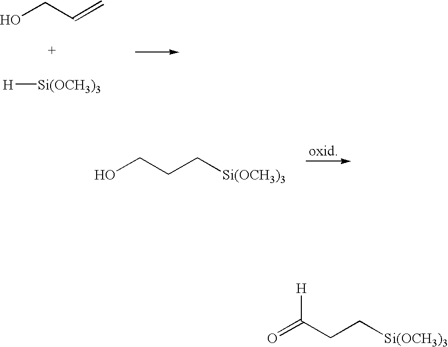

Another synthesis method is the thiol-ene addition:

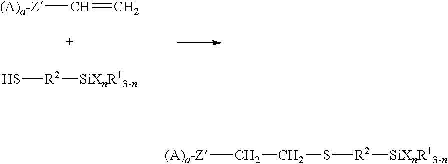

Specific example for the preparation of an aldehyde-group-containing silane: thiol-ene addition of acrolein with 3-mercaptopropyltrimethoxysilane:

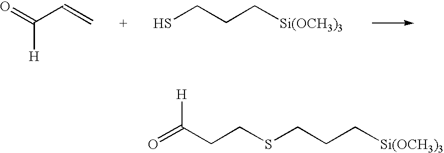

A further synthesis method is the addition of isocyanate groups:

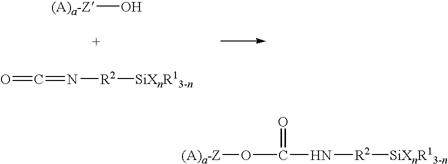

Specific example for the preparation of an aldehyde-group-containing silane: reaction of 4-hydroxymethylbenzaldehyde with 3-isocyanatopropyltriethoxysilane:

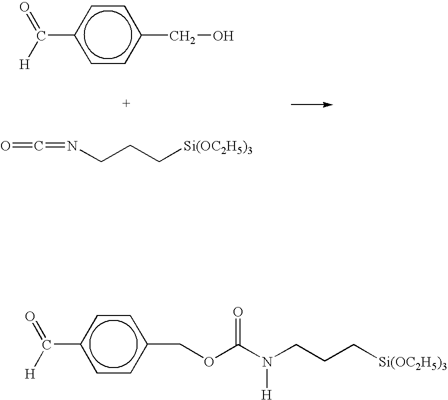

In addition to the exemplary synthesis methods mentioned above for the preparation of the silanes according to formula (II), further methods are generally known to a person skilled in the art.

Fillers surface-modified with groups of formula (I) can be obtained in particular by reaction of the filler with a silane. In the case of silicate fillers, stable siloxane bonds are thus formed between silanol groups on the surface of the filler and silicon atoms of the silane.

In one embodiment, a filler that is surface-modified with groups of formula (I) is obtained by reacting the filler with at least one silane of formula (II).

In another embodiment, in a first step the filler is reacted with a silane which represents a precursor of a silane of formula (II), and the obtained product is then converted in one or more steps into a filler that is surface-functionalized with groups of formula (I). Silanes which represent a precursor of a silane of formula (II) according to one of the processes discussed above are particularly preferred.

For example, in a first step a silylation of the filler with a hydrosilane of formula H—SiXₙR¹₃₋ₙ can take place and the thus-obtained product can then be reacted in a hydrosilylation with a vinyl-group-containing compound of formula (A)ₐ-Z-Y—R′—CH═CH₂. Specific example for the preparation of an aldehyde-group-containing filler: silylation of the filler with trimethoxysilane, followed by reaction of the product with allyl alcohol and finally oxidation of the primary OH group to give the aldehyde group.

According to another synthesis method, in a first step the filler is silanized with a mercaptoalkylsilane of formula HS—R²—SiXₙR¹₃₋ₙ and the thus-obtained product is then reacted in a thiol-ene addition with a vinyl-group-containing compound of formula (A)ₐ-Z′-CH═CH₂. Specific example for the preparation of an aldehyde-group-containing filler: silylation of the filler with 3-mercaptopropyltrimethoxysilane followed by reaction of the product with acrolein.

According to a further synthesis method, in a first step the filler is silanized with an isocyanatoalkylsilane of the formula O═C═N—R²—SiXₙR¹₃₋ₙ and the thus-obtained product is then reacted with an alcohol of formula (A)ₐ-Z-OH. Specific example for the preparation of an aldehyde-group-containing filler: silanization of the filler with 3-isocyanatopropyltriethoxysilane followed by reaction of the product with 4-hydroxymethylbenzaldehyde.

In the case of functionalization with phosphonic acid groups —P(O)(OH)₂ or dihydrogen phosphate groups —O—P(O)(OH)₂, one can also first perform a surface functionalization of the filler with a silane that contains at least one phosphonic acid ester group —P(O)(OR³)₂ or phosphoric acid ester group —O—P(O)(OR³)₂. Subsequently, liberation of the corresponding acid group(s) is achieved by hydrolysis or alcoholysis.

For example, in a first synthesis step a filler can be reacted with commercially available diethoxyphosphorylethyltriethoxysilane (R³=Et) for functionalization with phosphonic acid groups, whereby the silane is bonded to the filler. In the second step, the phosphonic acid group is then liberated by hydrolysis of the phosphonic acid ester group. By way of example, this is shown for the case of a silicate filler in the following diagram:

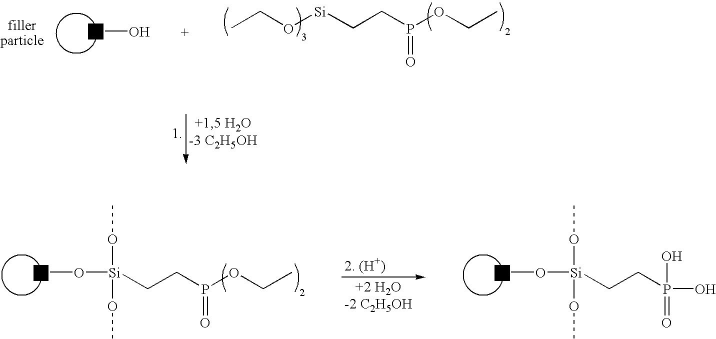

The preparation of a filler that is surface-functionalized with groups of formula (I) by reaction of a filler with a silane can be carried out in various ways. For example a liquid silane can be directly mixed with filler and can then be dried to separate off condensation products.

In another embodiment, a filler is dispersed in a solution of the silane in a suitable solvent. The interaction of the silane with the filler surface can be influenced by the polarity of the solvent. It has been found that such process provides for a better wetting of the filler surface and is advantageous in particular in the case of very fine-particle fillers with a specific surface greater than 30 m²/g, in particular greater than 40 m²/g. Examples of suitable solvents are C₁-C₆ alkanols, such as e.g. ethanol or isopropanol, cyclic ethers, such as e.g. tetrahydrofuran or dioxan, aliphatic esters, such as e.g. ethyl acetate or butyl acetate, aliphatic hydrocarbons, such as e.g. hexane, and cycloaliphatic hydrocarbons, such as e.g. cyclohexane.

After the reaction is complete, the filler is separated off, optionally washed one or more times with the same and/or another solvent, optionally subjected to a heat treatment, optionally washed again and then dried. After the surface functionalization, the filler is optionally ground. This can be advantageous in particular in the case of fillers which tend to agglomerate.

In particular when using nanoparticles as filler, it can be advantageous to use the filler in the form of an organosol. The term “organosol” here refers in particular to colloidal suspensions in which the continuous phase is an organic compound, in particular an organic solvent or a polymerizable monomer that is liquid at room temperature. Examples of suitable polymerizable monomers are as described below.

When reacting a filler with a silane, the degree of surface functionalization depends inter alia on the quantity of filler or the specific surface of the filler, on the quantity and structure of the silane, the reaction time, the temperature, the type of catalyst used and the filler pre-treatment, such as e.g. a pre-drying. The various influencing factors have generally been very well investigated particularly in the case of the silanization of SiO₂ (cf E. P. Plueddemann, “Silane Coupling Agents”, Plenum Press, 2^{nd} ed., New York and London, 1991; A. Guillet, Macromol. Symp. 194 (2003) 63), which is hereby incorporated by reference in its entirety).

According to the invention, fillers surface-functionalized with groups of formula (I) are preferred which can be obtained by reacting a filler with at least 0.01 mmol, preferably 0.1-5 mmol, particularly preferably 0.5 to 2 mmol of a suitable silane per gram of the filler. Silanes of formula (II) and silanes which represent a precursor of a silane of formula (II), as described above, are particularly preferred.

The degree of surface functionalization of the filler that is surface-functionalized with groups of formula (I) can be determined for example by means of elemental analysis. In the case of groups of formula (I) which contain phosphorous and/or sulphur, in particular the level of one of these elements in the surface-functionalized filler can be used to determine the degree of functionalization.

It is preferred that the filler that is surface-functionalized with groups of formula (I) contains at least 0.01 mmol, preferably 0.05-2 mmol, particularly preferably 0.1 to 1 mmol of groups of formula (I) per gram of the filler. In the case of fillers based on SiO₂, it is preferred in particular that the filler that is surface-functionalized with groups of formula (I) contains at least 0.01 mmol, preferably 0.05-2 mmol, particularly preferably 0.1 to 1 mmol of groups of formula (I) per gram of SiO₂.

In the compositions according to the invention, the filler that is surface-functionalized with groups of formula (I) can also be modified with further groups. The term “further group” here refers to a group which does not have the formula (I). For example, the filler that is surface-functionalized with groups of formula (I) can additionally be modified with polymerizable and/or non-functionalized groups. Preferred polymerizable groups are groups which have at least one (meth)acrylic ester and/or (meth)acrylamide functionality, in particular (meth)acryloyloxyalkylsilyl groups or (meth)acrylamidoalkylsilyl groups. By alkyl is preferably meant radicals with 1 to 6, in particular 1 to 3 carbon atoms. By non-functionalized groups is meant groups which do not have the formula (I) and which are not polymerizable. An additional surface modification of the filler, for example with polymerizable groups, can in particular improve the incorporability of the filler into the compositions according to the invention and the mechanical properties of dental materials prepared therefrom.

Fillers that are surface-functionalized with groups of formula (I) which are additionally modified with further groups can be obtained in particular by reacting the filler before, after or together with the surface functionalization with at least one group of formula (I) with at least one further silane. Preferably, a mixture of at least one silane of formula (II) with at least one further silane is used in the surface functionalization of the filler. According to another variant, before or after surface modification of a filler with at least one silane of formula (II), a silanization of the filler with one or more polymerizable and/or non-functionalized further silanes is performed.

Examples of suitable polymerizable silanes are (meth)acryloyloxyalkyltrialkoxysilanes, such as e.g. 3-methacryloyloxypropyltrimethoxysilane, 3-acryloyloxypropyltrimethoxysilane, 3-methacryloyloxypropylmethyldimethoxysilane, 3-methacryloyloxypropyldimethylmethoxysilane, 3-acryloyloxypropylmethyldimethoxysilane or 3-acryloyloxypropyldimethylmethoxysilane. Polymerizable silanes which carry two methacrylate radicals can easily be prepared e.g. by reaction of glycerol dimethacrylate with 3-isocyanatopropyltriethoxysilane or 3-(methyldiethoxysilyl)-propylsuccinic acid anhydride or with glutaric acid anhydride and then with 3-aminopropyltriethoxysilane. Also suitable are polymerizable (meth)acrylamidoalkyltrialkoxysilanes, such as e.g. 3-(N-methacryloylamino)-propyltrimethoxysilane, 3-(N-acryloylamino)-propyltrimethoxysilane, 3-(N-methacryloylamino)-propyltriethoxysilane, 3-(N-methacryloyl-N-ethyl-amino)-propyltrimethoxysilane, 3-(N-methacryloyl-N-ethyl-amino)-propyltrimethoxysilane, 3-(N-acryloyl-N-ethyl-amino)-propyltrimethoxysilane, 3-(N-methacryloyl-N-methyl-amino)-propyltrimethoxysilane or 3-(N-acryloyl-N-methyl-amino)-propyltrimethoxysilane, the polymerizable (meth)acrylamide groups of which have particularly good hydrolysis stability.

In addition to a filler that is surface-functionalized with groups of formula (I), the compositions according to the invention contain at least one polymerizable monomer. In particular, radically polymerizable monomers are suitable as polymerizable monomers.

These radically polymerizable monomers can have one or more radically polymerizable groups. Preferred radically polymerizable monomers are monomers which are liquid at room temperature and which are suitable as diluting monomers. Monomers having a viscosity of 0.01 to 10 Pa·s at room temperature, in particular mono- or polyfunctional (meth)acrylates, are preferred. Particularly preferred are hydrolysis-resistant diluting monomers, in particular mono(meth)acrylates, such as e.g. mesityl methacrylate, 2-(alkoxymethyl)acrylic acids, such as e.g. 2-(ethoxymethyl)acrylic acid and 2-(hydroxymethyl)acrylic acid, N-mono-alkyl-substituted acrylamides, such as e.g. N-ethylacrylamide or N-(2-hydroxyethyl)acrylamide, N-mono-alkyl-substituted methacrylamides, such as e.g. N-ethylmethacrylamide, N-(2-hydroxyethyl)methacrylamide or N-(5-hydroxypentyl)methacrylamide, N,N-dialkyl-substituted acrylamides, such as e.g. N,N-dimethylacrylamide or N-methyl-N-(2-hydroxyethyl)acrylamide, and N-vinyl pyrrolidone. By alkyl is preferably meant radicals with 1 to 6, in particular 1 to 3 carbon atoms. Examples of further diluting monomers are mono(meth)acrylates, such as e.g. methyl, ethyl, butyl, benzyl, furfuryl or phenyl(meth)acrylate.

In addition to the filler that is surface-functionalized with groups of formula (I), the compositions according to the invention preferably contain 0 to 50 wt.-%, preferably 5 to 40 wt.-% and quite particularly preferably 10 to 30 wt.-% diluting monomer. These and, unless otherwise stated, all other percentages relate to the overall mass of the composition.

The compositions according to the invention preferably contain at least one monomer with 2 or more, in particular 2 to 5 radically polymerizable groups. Monomers with 2 or more polymerizable groups act as crosslinkers and thus increase the mechanical stability of the cured compositions.

Preferred crosslinking monomers are hydrolysis-resistant crosslinking monomers, in particular crosslinking pyrrolidones, such as e.g. 1,6-bis(3-vinyl-2-pyrrolidonyl)-hexane or commercially available bis(meth)acrylamides, such as e.g. methylene or ethylene bisacrylamide, N,N′-diethyl-1,3-bis(acrylamido)-propane, 1,3-bis(methacrylamido)-propane, 1,4-bis(acrylamido)-butane, 1,4-bis(acryloyl)-piperazine, 2,6-dimethylene-4-oxa-heptane-1,7-dicarboxylic acid-bis-(propylamide), 1,6-bis-(acrylamido)-2,2,4(2,4,4)-trimethylhexane and N,N′-dimethyl-1,6-bis-(acrylamido)-hexane. Examples of further crosslinkers are polyfunctional (meth)acrylates, such as e.g. bisphenol-A-di(meth)acrylate, bis-GMA (an addition product of methacrylic acid and bisphenol-A-diglycidyl ether), UDMA (an addition product of 2-hydroxyethyl methacrylate and 2,2,4-hexamethylene diisocyanate), di-, tri- or tetraethylene glycol di(meth)acrylate, trimethylolpropane tri(meth)acrylate, pentaerythritol tetra(meth)acrylate, butanediol di(meth)acrylate, 1,10-decanediol di(meth)acrylate or 1,12-dodecanediol di(meth)acrylate.

Compositions which, in addition to the filler that is surface-functionalized with groups of formula (I), contain 0 to 45 wt.-%, preferably 1 to 30 wt.-% and quite particularly preferably 5 to 20 wt.-% crosslinking monomer, in particular bis(meth)acrylamide, are particularly preferred according to the invention.

According to a further preferred embodiment, the compositions contain at least one acidic radically polymerizable monomer, i.e. a monomer with one or more acidic groups, such as carboxylic acid anhydride, carboxylic acid, phosphoric acid, dihydrogen phosphate, phosphonic acid and sulphonic acid groups. Preferred acidic groups are carboxylic acid, phosphoric acid and phosphonic acid groups. Such monomers are suitable as adhesive monomers in particular for enamel/dentine adhesives or self-adhesive composites.

Particularly preferred acidic monomers are polymerizable acrylate ether phosphonic acids, such as e.g. 2-[4-(dihydroxyphosphoryl)-2-oxa-butyl]-acrylic acid ethyl ester, 2-[4-(dihydroxyphosphoryl)-2-oxa-butyl]-acrylic acid or 2-[4-(dihydroxyphosphoryl)-2-oxa-butyl]-acrylic acid-2,4,6-trimethyl phenyl ester, (meth)acrylamidoalkylene phosphonic acids or (meth)acrylamidoalkylene bisphosphonic acids. Furthermore, hydrolysis-resistant, polymerizable dihydrogen phosphates such as (meth)acrylamidoalkylene phosphates, (meth)acrylamidocycloalkylene phosphates or (meth)acrylamidoarylene dihydrogen phosphates, e.g. 2-(N-acryloylamino)ethyl dihydrogen phosphate, 2-(N-methacryloylamino)ethyl dihydrogen phosphate, 6-(N-acryloylamino)hexyl dihydrogen phosphate, 6-(N-methacryloylamino)hexyl dihydrogen phosphate, 4-(N-acryloylamino)phenyl dihydrogen phosphate, 4-(N-methacryloylamino)phenyl dihydrogen phosphate, 1,3-bis-(N-acryloylamino)-propan-2-yl-dihydrogen phosphate, 1,3-bis-(N-methacryloylamino)-propan-2-yl-dihydrogen phosphate, 1,3-bis-(N-acryloyl-N-methyl-amino)-propan-2-yl-dihydrogen phosphate or 1,3-bis-(N-acryloyl-N-ethyl-amino)-propan-2-yl-dihydrogen phosphate are also particularly suitable as adhesive monomers.

Compositions which, in addition to the filler that is surface-functionalized with groups of formula (I), contain 1 to 50 wt.-%, preferably 5 to 40 wt.-% and quite particularly preferably 10 to 30 wt.-% acidic monomer, in particular acidic monomer with dihydrogen phosphate, phosphonic acid and/or sulphonic acid groups, are particularly preferred according to the invention.

In addition to the filler that is surface-functionalized with groups of formula (I), the composition according to the invention can preferably contain at least one further filler that is not surface-modified with groups of formula (I). Examples of suitable further fillers are fillers which are not surface-modified, fillers which are surface-modified with polymerizable groups, and fillers which are surface-modified with non-functionalized groups. Preferred polymerizable groups are groups which have at least one (meth)acrylic ester and/or (meth)acrylamide functionality, in particular (meth)acryloyloxyalkylsilyl groups or (meth)acrylamidoalkylsilyl groups. By alkyl is preferably meant radicals with 1 to 6, in particular 1 to 3 carbon atoms. By non-functionalized groups is meant groups which do not have the formula (I) and are not polymerizable. Such surface-modified fillers can be obtained in particular by silanization of a filler with suitable silanes. For example, fillers which are surface-modified with polymerizable groups can be obtained by silanization of a filler with at least one of the polymerizable silanes described above.

In addition to the filler that is surface-functionalized with groups of formula (I), the composition according to the invention preferably contains 0 to 40 wt.-%, in particular 1 to 30 wt.-% further filler that is not surface-modified with groups of formula (I).

To initiate the polymerization, the compositions according to the invention preferably contain an initiator for radical polymerization, in particular for photochemical or redox-induced radical polymerization. Examples of suitable initiators for photopolymerization are benzophenone, benzoin and derivatives thereof or a-diketones or derivatives thereof, such as 9,10-phenanthrenequinone, 1-phenyl-propan-1,2-dione, diacetyl or 4,4′-dichlorobenzil. It is particularly preferred to use camphorquinone and 2,2-dimethoxy-2-phenyl-acetophenone, and it is quite particularly preferred to use α-diketones in combination with amines as reducing agents. Preferred amines are 4-(N,N-dimethylamino)-benzoic acid ester, N,N-dimethylaminoethylmethacrylate, N,N-dimethyl-sym.-xylidine and triethanolamine. In addition, acylphosphines, such as e.g. 2,4,6-trimethylbenzoyldiphenyl or bis-(2,6-dichlorobenzoyl)-4-N-propylphenylphosphine oxide are particularly suitable.

Redox-initiator combinations, such as e.g. combinations of benzoyl peroxide with N,N-dimethyl-sym.-xylidine or N,N-dimethyl-p-toluidine, are used as initiators for a polymerization carried out at room temperature. In addition, redox systems consisting of peroxides and reducing agents, such as e.g. ascorbic acid, barbiturates or sulphinic acids, are also particularly suitable.

Compositions which, in addition to the surface-functionalized filler, contain 0.01 to 5.0 wt.-%, preferably 0.2 to 2.0 wt.-% and quite particularly preferably 0.2 to 1.0 wt.-% initiator for the radical polymerization, are particularly preferred according to the invention.

The compositions according to the invention can further contain solvents, such as water, ethyl acetate or ethanol, or solvent mixtures. Hydrolysis-resistant solvents, such as water or ethanol, or solvent mixtures are preferred.

In addition, the compositions according to the invention can contain further additives, in particular stabilizers, flavourings, dyes, microbiocidal active agents, fluoride-ion-releasing additives, optical brighteners, plasticizers and UV absorbers.

Compositions which contain the following components are preferred according to the invention:
a) 1 to 60 wt.-%, in particular 5 to 40 wt.-% filler that is surface-functionalized with groups of formula (I),
b) 0 to 40 wt.-%, in particular 0 to 30 wt.-% filler that is not surface-functionalized with groups of formula (I),
c) 0 to 70 wt.-%, in particular 1 to 40 wt.-% diluting and/or crosslinking monomer,
d) 0.01 to 5.0 wt.-%, in particular 0.2 to 2.0 wt.-%, particularly preferably 0.2 to 1.0 wt.-% initiator for the radical polymerization,
e) 0 to 70 wt.-%, in particular 0 to 50 wt.-% acidic monomer and/or solvent.

All percentages relate to the overall mass of the composition. Compositions which contain at least one acidic monomer or at least one crosslinking monomer, in particular at least one acidic monomer and at least one crosslinking monomer or at least one acidic crosslinking monomer, are quite particularly preferred.

The compositions according to the invention are particularly suitable as dental materials, in particular as adhesives, cements, preferably self-adhesive cements such as e.g. fixing cements, and composites, preferably filling composites. Such dental materials are characterized by a very good adhesion to the tooth structure, i.e. to enamel and dentine.

The preferred compositions according to the invention cure with formation of strongly crosslinked polymer networks which swell little or not at all in water.

The invention also relates to a surface-functionalized filler as defined above. The invention furthermore also relates to a process for the preparation of a composition according to the invention or of a surface-functionalized filler according to the invention, wherein the filler is reacted with at least one silane and the obtained surface-functionalized filler is mixed with the further constituents of the composition. Preferred embodiments of the reaction of the filler with at least one silane are as described above.

Finally, the invention also relates to the use of a filler that is surface-functionalized with groups of formula (I) for the preparation of a dental material, in particular an adhesive or cement.

The invention is explained in more detail below by means of examples.

### EXAMPLES

### Example 1

### Surface Functionalization of SiO₂ Nanoparticles (d=13 nm) of an SiO₂ Organosol With Aldehyde Groups

### 1^{st} Stage: Functionalization of SiO₂ Nanoparticles With SH Groups

2.12 g 3-mercaptopropyltrimethoxysilane (10.8 mmol) was added to 60.0 g SiO₂ organosol (NanO G 502-31, Clariant; 30 wt.-% SiO₂ in isopropanol). Subsequently, 0.584 g (32.4 mmol based on water) of a 0.5 N HCl solution was added and stirred for 48 h at room temperature. The dispersion was then cooled to 0° C. and 0.98 g (9.0 mmol) chlorotrimethylsilane was added dropwise. After stirring for 24 h at room temperature, 40 ml toluene was added and the isopropanol and the formed methanol were distilled off at 40° C. and 140 mbar-80 mbar. Toluene that had also distilled off was subsequently added again, so that the concentration of SiO₂ in the dispersion did not exceed 30.0 wt.-%. 75.3 g of a slightly viscous, cloudy and thixotropic liquid was obtained.

### 2^{nd} Stage: Thiol-Ene Addition of Acrolein to SH-Functionalized SiO₂ Nanoparticles

0.605 g (10.8 mmol) acrolein and a spatula-point of hydroquinone were introduced into a flask under argon. 75.3 g of the organosol prepared in the 1^{st} stage was then added dropwise at 0 to 5° C. within 11 h. The ice bath was removed and the dispersion was stirred for 1 week at room temperature. In the obtained organosol, no acrolein was present any more. The product contained approximately 58% aldehyde groups, relative to the trialkoxysilane used. The residue on ignition of the slightly viscous, cloudy and thixotropic organosol was 7.1% SiO₂.

### Example 2

### Preparation of an SiO₂ Organosol Surface-Functionalized With Aldehyde Groups With a Radically Crosslinkable Dispersing Agent

25.0 g N,N′-diethyl-1,3-bis(acrylamido)-propane was dissolved in 70.7 g of the SiO₂ organosol functionalized with aldehyde groups from the 2^{nd} stage of Example 1 (SiO₂ content: 5 g). A translucent solution formed. The toluene was then removed on the rotary evaporator at 40° C. A light brownish, translucent-cloudy liquid with a viscosity of 7.0-5.5 Pa·s (1-100 s⁻¹) and a residue on ignition of 15.8% was obtained which was able to be cured to form an insoluble solid after the addition of a radical initiator.

### Example 3

### Surface Functionalization of Pyrogenic Silicic Acid With Aldehyde Groups

### 1^{st} Stage: Functionalization of Pyrogenic Silicic Acid OX-50 With SH Groups

20.0 g pyrogenic silicic acid OX-50 was suspended in 200 g ethanol. 0.56 g of a 0.5 N HCl solution was then added and the suspension was heated to 70° C. 2.04 g (10.4 mmol) 3-mercaptopropyltrimethoxysilane was added, and the suspension was stirred for 30 h at 70° C. The solvent was then removed on the rotary evaporator at 40° C. The obtained white powder was dispersed in 40 g acetone and then separated off again by centrifugation. The powder was then dispersed in 50 g ethanol, centrifuged off, finally dispersed in cyclohexane and centrifuged off again. The obtained powder was dried on the rotary evaporator at 8·10⁻² mbar. The mercapto group coverage was 0.12 mmol/g SiO₂ and was determined via the sulphur content (0.38 wt.-%, elemental analysis) of the sample.

### 2^{nd} Stage: Thiol-Ene Addition of Acrolein to SH-Functionalized SiO₂ Particles

10.0 g functionalized particles from the 1^{st} stage was dispersed in 30 g toluene under ultrasound. Subsequently, 0.24 g (4.3 mmol) acrolein was added dropwise and the suspension was stirred for 48 h at 50° C. Then, the solvent and unreacted acrolein were removed on the rotary evaporator at 40° C. and the powder was dried at 8·10⁻² mbar. The functionalized silane coverage was 0.10 mmol/g SiO₂ and was determined via the sulphur content (0.33 wt.-%, elemental analysis) of the sample.

### Example 4

### Surface Functionalization of Pyrogenic Silicic Acid (Aerosil 200) With Phosphonic Acid Groups

### 1^{st} Stage: Functionalization of Pyrogenic Silicic Acid With Diethyl Phosphonate Groups

25.0 g Aerosil 200 was suspended in 750 g cyclohexane. 13.6 g (41.4 mmol) diethylphosphorylethyltriethoxysilane and 3.68 g (62.25 mmol) n-propylamine were then added. The mixture was stirred for 30 h at 70° C. The solvent was removed on the rotary evaporator at 40° C. and the product was dried for an additional 3 days at 50° C. in a drying oven. The powder was then suspended in 150 ml ethanol for washing and was separated from the solvent by means of pressure filtration (0.45 μm). The powder was washed once more analogously with ethanol and then once with cyclohexane. Drying was then carried out for a further 3 days at 50° C. in a drying oven. The residue on ignition was 93.1 wt.-%. The functionalized silane coverage was 0.66 mmol/g SiO₂ and was determined via the phosphorous content (1.78 wt.-%, elemental analysis) of the sample.

### 2^{nd} Stage: Liberation of the Phosphonic Acid Groups

25.0 g silanized Aerosil 200 from the 1^{st} stage was suspended in 460 g hydrochloric acid (32 wt.-%) and heated under reflux for 46 h. The hydrochloric acid solution was then removed under vacuum at 40° C. and the powder was dried for 3 days in a drying oven at 50° C. The modified particles were then redispersed in 150 ml water and filtered by means of pressure filtration (0.45 μm). The process was repeated twice more and the powder was then dried for 3 days in a drying oven at 50° C. The residue on ignition was 96.0 wt.-%. The functionalized silane coverage was 0.58 mmol/g SiO₂ and was determined via the phosphorous content (1.85 wt.-%, elemental analysis) of the sample.

### Example 5

### Additional Surface Functionalization of Pyrogenic Silicic Acid Modified With Phosphonic Acid Groups With Methacrylate Groups

11.0 g modified Aerosil from the 2^{nd} stage of Example 4 was suspended in 330 g n-hexane. 1.99 g (9.0 mmol) 3-methacryloyloxypropyldimethylchlorsilane (ABCR), dissolved in 30 ml n-hexane, was then slowly added dropwise. The mixture was stirred at room temperature for a further 47 h. The solvent was then removed under vacuum at 40° C., and the powder was dried for 3 days in a drying oven at 50° C. The particles were dispersed in 150 ml ethanol and filtered by means of pressure filtration (0.45 μm), dispersed in ethanol and filtered a second time and finally dispersed in 150 ml cyclohexane and filtered off once more. The powder was dried for 3 days in a drying oven at 50° C. The residue on ignition was 94.8 wt.-%. it was possible to verify the presence of methacrylate groups by means of IR spectroscopy by the appearance of a new band at 1636 cm⁻¹.

### Example 6

### Surface Functionalization of Pyrogenic Silicic Acid (Aerosil Ox-50) With Phosphonic Acid Groups

### 1^{st} Stage: Functionalization of Pyrogenic Silicic Acid With Diethylphosphonate Groups

20.0 g pyrogenic silicic acid OX-50 was suspended in 200 g ethanol. Subsequently, first 3.41 g (10.4 mmol) diethylphosphorylethyltriethoxysilane and then 0.56 g of a 0.5 N hydrochloric acid solution were added. The suspension was heated to 70° C. and stirred at this temperature for 30 h. The volatile constituents were then removed on the rotary evaporator at 40° C. and the powder was dried for 3 days at 50° C. in a drying oven. The powder was then suspended for washing in 50 ml ethanol and separated from the solvent by means of centrifugation (up to 5000 rpm). The powder was washed once more analogously with ethanol and then once with 50 ml cyclohexane. Drying was then carried out for a further 3 days at 50° C. in a drying oven. The functionalized silane coverage was 0.11 mmol/g SiO₂ and was determined via the phosphorous content (0.32 wt.-%, elemental analysis) of the sample.

### 2^{nd} Stage: Liberation of the Phosphonic Acid Groups

10.0 g modified OX-50 from the 1^{st} stage was dispersed in 100 g hydrochloric acid (32 wt.-%) and heated under reflux at 100° C. for 24 h. The hydrochloric acid was then removed under vacuum on the rotary evaporator at 40° C. The powder was then dried for 3 days in a drying oven at 50° C. The modified particles were then redispersed in 50 ml water and separated off again by means of centrifugation. The process was repeated twice more and the powder was then dried for 3 days in a drying oven at 50° C. The functionalized silane coverage was 0.12 mmol/g SiO₂ and was determined via the phosphorous content (0.36 wt.-%, elemental analysis) of the sample.

### Example 7

### Enamel-Dentine Adhesive Which Contains Aldehyde-Functionalized Sio₂ Particles

To examine the enamel and dentine adhesion, the adhesives A (according to the invention) and B (comparative example) were prepared by mixing the starting components with the composition shown in the table below (details in wt.-%). The adhesion of the adhesives to tooth enamel and dentine was determined.

For this, bovine teeth were embedded in plastic cylinders such that the dentine or the tooth enamel and the plastic were on one plane. After grinding of the testpieces, a layer of adhesive of the above formulation was massaged onto the dentine surface with a microbrush for 30 s, blown on briefly with an air brush and lit for 20 s with an Astralis 7 photopolymerization lamp (Ivoclar Vivadent AG). The filling composite Tetric® Ceram (Ivoclar Vivadent AG) was then applied to the adhesive layer and cured for 40 s with the Astralis 7 lamp. The testpieces were then stored in water for 24 h at 37° C. and the adhesive shear strength was measured according to the ISO guideline “ISO 1994-ISO TR 11405: Dental Materials Guidance on Testing of Adhesion to Tooth Structure”. The results show that the aldehyde-functionalized particles lead to an improvement in the adhesive shear strength to dentine and tooth enamel.

| | adhesion values | |
|---|---|---|
| | Adhesive A | |
| | (according to the | Adhesive B |
| Components | invention) | (comparative) |
| Bismethacrylamide phosphate¹⁾ | 14.6 | 14.6 |
| 2-(acryloylamino)-succinic acid | 9.7 | 9.7 |
| Water | 23.0 | 23.0 |
| Aerosil 200, unmodified | — | 3 |
| SiO₂ particles from Example 2²⁾ | 2.4 | — |
| N,N′-diethyl-1,3-bis(acrylamido)- | 47.6 | 47.0 |
| propane³⁾ | | |
| N-(5-hydroxypentyl)methacrylamide | 2.0 | 2.0 |
| Photoinitiator⁴⁾ | 0.7 | 0.7 |
| Adhesion to enamel (MPa) | 21 | 16 |
| Dentine adhesion (MPa) | 23 | 21 |

¹⁾1,3-bis-(N-methacryloylamino)-propan-2-yl-dihydrogen phosphate
²⁾worked in as organosol, value excluding the N,N′-diethyl-1,3-bis(acrylamido)-propane contained in the organosol
³⁾in the case of adhesive A, including the quantity contained in the organosol used
⁴⁾photoinitiator: 0.3 wt.-% camphorquinone, 0.4 wt.-% 4-dimethylaminobenzoic acid ethyl ester

Although various embodiments have been depicted and described in detail herein, it will be apparent to those skilled in the relevant art that various modifications, additions, substitutions, and the like can be made without departing from the spirit of the invention and these are therefore considered to be within the scope of the invention as defined in the claims which follow.

What is claimed:

1. A polymerizable composition comprising at least one filler that is surface-functionalized with groups of formula (I), wherein groups of formula (I)
(A)ₐ-Z-Y—R²—SiR¹₃₋ₘ—(O—)ₘ (I),
in which
R¹ represents C₁-C₁₅ alkyl, C₂-C₅ alkenyl or phenyl,
R² is missing or represents unbranched or branched C₁-C₆ alkylene,
Y is missing or represents an ether, thioether, amide, ester or urethane group,
Z is missing or represents an at least divalent linear or branched aliphatic radical with 2 to 40 carbon atoms, which can be interrupted by one or more ether, thioether, amide or ester groups and which can contain one or more cycloaliphatic groups with at least 3 carbon atoms and/or one or more aromatic groups with at least 6 carbon atoms, an at least divalent cycloaliphatic radical with at least 3 carbon atoms or an at least divalent aromatic radical with at least 6 carbon atoms,
A represents in each independently —COOH, —P(O)(OH)₂, —O—P(O)(OH)₂, —SO₂OH, —C(O)—O—C(O)—, —CHO, —NH—C(O)—CHO, —C(O)—CHO, —C(O)—CH₂—C(O)—CH₃, —N═C═O or —O—C(O)—CH₂—C(O)—CH₃,
a is 1 to 6 and
m is 1 to 3,
wherein R² and Z cannot both be missing,
wherein R² and Z can in each case be missing only if Y is simultaneously also missing, and
wherein a is 1 if Z is missing,
are bonded to the filler via at least one oxygen atom that is bound to the silicon atom of the group of formula (I).

2. The polymerizable composition according to claim 1, wherein
R¹ represents C₁-C₆ alkyl or phenyl,
R² represents linear or branched C₁-C₃ alkylene,
Y is th missing or represents an ether, thioether, ester or urethane group,
Z is missing or represents an at least divalent linear or branched aliphatic radical with 2 to 20 carbon atoms, which can be interrupted by one or more ether, thioether, amide or ester groups and which can contain one or more cycloaliphatic groups with at least 3 carbon atoms and/or one or more aromatic groups with at least 6 carbon atoms, an at least divalent cycloaliphatic radical with at least 3 carbon atoms or an at least divalent aromatic radical with at least 6 carbon atoms,
A represents in each case independently —COOH, —P(O)(OH)₂, —O—P(O)(OH)₂, —SO₂OH, —CHO, —NH—C(O)—CHO or —O—C(O)—CH₂—C(O)—CH₃,
a is 1 to 3 and
m is 1 to 3.

3. The polymerizable composition according to claim 1, wherein the filler is a particulate filler with an average particle size of from 1 nm to 10 μm.

4. The polymerizable composition according to claim 1, wherein the filler is a monodisperse, nanoparticulate filler based on SiO₂, oxides of the elements Zr, Ti, Al, Y, La, Ce and/or Yb or their mixed oxides with SiO₂.

5. The polymerizable composition according to claim 1, wherein the filler has an average particle size of 5 to 200 nm.

6. The polymerizable composition according to claim 1, wherein the filler that is surface-functionalized with groups of formula (I) contains at least 0.01 mmol groups of formula (I) per gram of the filler.

7. The polymerizable composition according to claim 1, wherein the filler is additionally surface-modified with at least one further group.

8. The polymerizable composition according to claim 1, wherein the composition contains at least one radically polymerizable monomer.

9. The polymerizable composition according to claim 1, wherein the composition contains an initiator for radical polymerization.

10. The polymerizable composition according to claim 1, wherein the composition contains at least one monomer with 2 or more polymerizable groups and/or at least one monomer with one or more acidic groups.

11. The polymerizable composition according to claim 9, wherein the composition contains an initiator for photopolymerization.

12. The polymerizable composition according to claim 1, further comprising a filler that is not surface-functionalized with groups of formula (I).

13. The polymerizable composition according to claim 1, comprising
a) 1 to 60 wt.-% filler that is surface-functionalized with groups of formula (I),
b) 1 to 40 wt.-% filler that is not surface-functionalized with groups of formula (I),
c) 0 to 70 wt.-% diluting and/or crosslinking monomer,
d) 0.1 to 5.0 wt.-% initiator for radical polymerization,
e) 0 to 70 wt.-% acidic monomer and/or solvent.

14. A surface-functionalized filler, comprising groups of formula (I),
(A)ₐ-Z-Y—R²—SiR¹₃₋ₘ—(O—)ₘ (I),
in which
R¹ represents C₁-C₁₅ alkyl, C₂-C₅ alkenyl or phenyl,
R² is missing or represents unbranched or branched C₁-C₆ alkylene,
Y is missing or represents an ether, thioether, amide, ester or urethane group,
Z is missing or represents an at least divalent linear or branched aliphatic radical with 2 to 40 carbon atoms, which can be interrupted by one or more ether, thioether, amide or ester groups and which can contain one or more cycloaliphatic groups with at least 3 carbon atoms and/or one or more aromatic groups with at least 6 carbon atoms, an at least divalent cycloaliphatic radical with at least 3 carbon atoms or an at least divalent aromatic radical with at least 6 carbon atoms,
A represents in each case independently —COOH, —P(O)(OH)₂, —O—P(O)(OH)₂, —SO₂OH, —C(O)—O—C(O)—, —CHO, —NH—C(O)—CHO, —C(O)—CHO, —C(O)—CH₂—C(O)—CH₃, —N═C═O or —O—C(O)—CH₂—C(O)—CH₃,
a is 1 to 6 and
m is 1 to 3,
wherein R² and Z cannot both be missing,
wherein R² and Z can in each case be missing only if Y is simultaneously also missing, and
wherein a is 1 if Z is missing,
are bonded to the filler via at least one oxygen atom that is bound to the silicon atom of the group of formula (I).

15. A process for the preparation of a composition according to claim 1, comprising reacting a filler with at least one silane and mixing the obtained surface-functionalized filler with further constituents of the composition.

16. The process according to claim 15, wherein the silane has the formula (II)
(A)ₐ-Z-Y—R²—SiXₙR¹₃₋ₙ (II),
in which
X represents halogen, hydroxy, C₁-C₅-alkoxy or C₁-C₃-acyloxy and
n is 1 to 3.

17. The process according to claim 15, wherein
a) the silane is mixed in liquid form with the filler and
b) the filler is dried to separate off condensation products.

18. The process according to claim 15, wherein
a) the filler is dispersed in a solution of the silane in a solvent and
b) the filler is separated off and optionally washed one or more times with the solvent from step (a) and/or at least one other solvent,
c) the filler is optionally subjected to a heat treatment and optionally washed again,
d) the filler is dried and
e) the filler is optionally ground.

19. The polymerizable composition, which is obtainable by a process according to claim 15.

20. The surface-functionalized filler, which is obtainable by a process according to claim 15.

21. The polymerizable composition according to claim 5, wherein the filler has an average particle size of 10 to 100 nm.

22. The polymerizable composition according to claim 21, wherein the filler has an average particle size of 10 to 50 nm.

23. The polymerizable composition according to claim 6, wherein the filler contains 0.05 to 2 mmol groups of formula (I) per gram of the filler.

24. The polymerizable composition according to claim 23, wherein the filler contains 0.01 to 1 mmol groups of formula (I) per gram of the filler.
